SEQUENCE LISTING (13 sequences)

SEQ ID NO: 1 - DNA, 28 bp, Artificial
  PCR amplification primer for CMVp/TetR cassette
gcggccgctagggcctctgagctattcc

SEQ ID NO: 2 - DNA, 51 bp, Artificial
  PCR amplification primer for CMVp/TetR cassette
gaattctctgctttaataggatctgaactcccgggaaccgctgtacgcgga

SEQ ID NO: 3 - DNA, 35 bp, Artificial
  site-directed mutagenesis primer for mutating BglII site to BamHI site
cccaaagaagacaggatccgctttttgcctgtact

SEQ ID NO: 4 - DNA, 35 bp, Artificial
  site-directed mutagenesis primer for mutating BglII site to BamHI site
agtacaggcaaaaagcggatcctgtcttctttggg

SEQ ID NO: 5 - DNA, 23 bp, Artificial
  primer for tetO sequence insertion into mU6 promoter between PSE and TATA elements
ggatccgacgccgccatctctag

SEQ ID NO: 6 - DNA, 70 bp, Artificial
  primer for tetO sequence insertion into mU6 promoter between PSE and TATA elements
aaacaaggcttttctccaagggatatttataactctatcaatgatagagtactttacagttagggtgagt

SEQ ID NO: 7 - DNA, 20 bp, Artificial
  universal 5′ primer for tetO-mUS-siRNA cassette construction by PCR
gaactagtggatccgacgcc

SEQ ID NO: 8 - DNA, 16 bp, Artificial
  portion of siRNA-specific 3′ primer for tetO-mUS-siRNA cassette construction by PCR
tgctggatccaaaaaa

SEQ ID NO: 9 - DNA, 9 bp, Artificial
  portion of siRNA-specific 3′ primer for tetO-mUS-siRNA cassette construction by PCR
tctcttgaa

SEQ ID NO: 10 - DNA, 22 bp, Artificial
  portion of siRNA-specific 3′ primer for tetO-mUS-siRNA cassette construction by PCR
aaacaaggcttttctccaaggg

SEQ ID NO: 11 - DNA, 85 bp, Artificial
  siRNA-specific 3′ primer for MAP3K12
tgctggatccaaaaaagtcagaaacgtggcatctctctcttgaagagatgccacgtttctgacaaacaaggcttttctccaaggg

SEQ ID NO: 12 - DNA, 85 bp, Artificial
  siRNA-specific 3′ primer for mTOR
tgctggatccaaaaaagagaagaaatggaagaaattctcttgaaatttcttccatttcttctcaaacaaggcttttctccaaggg

SEQ ID NO: 13 - DNA, 89 bp, Artificial
  siRNA-specific 3′ primer for control (CNTL)
tgctggatccaaaaaaggcgcgctttgtaggattcgctctcttgaagcgaatcctacaaagcgcgccaaacaaggcttttctccaaggg

(19) United States
(12) Patent Application Publication
(10) Pub. No.: US 2009/0133136 A1
(43) Pub. Date: May 21, 2009

(54) Inducible SIRNA expression cassette and method of use

(75) Inventors: Demin Zhou, San Diego, CA (US); Jon E. Chatterton, Crowley, TX (US); Ning Ke, San Diego, CA (US); Jing Zhang, San Diego, CA (US); Joshua Bliesath, San Diego, CA (US); Henry Li, Carlsbad, CA (US); Flossie Wong-Staal, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Immusol Incorporated, San Diego, CA (US)

(21) Appl. No.: 11/814,158

(22) PCT Filed: Jan. 18, 2006
(86) PCT No.: PCT/US06/01997

§ 371 Date: Aug. 29, 2008

Related U.S. Application Data
(60) Provisional application No. 60/645,934, filed on Jan. 21, 2005.

(51) Int. Cl. A01K 67/027; C12N 15/82; C12N 15/85; A61K 48/00; C40B 40/06; C12N 5/10

(52) U.S. Cl. 800/14; 435/455; 435/468; 424/93.21; 435/320.1; 506/16; 435/325

(57) ABSTRACT

An inducible siRNA expression polynucleotide and methods for its use are provided. The expression polynucleotide comprises a bicistronic expression cassette that encodes a repressor and a detectable marker, wherein the repressor controls expression of siRNA expression in the absence of an inducer.

### BACKGROUND OF THE INVENTION

Cancer drug target validation is a crucial step toward developing an effective target-based cancer therapy. Since most therapeutics for cancer, either small molecule or antibody, are antagonists (loss of function), the drug targets are most likely proteins encoded by oncogenes. This inactivation of oncogenes leads to reduced transformation phenotypes of cancer cells, including growth arrest, reduced proliferation rate, decreased clonogenicity, and/or increased apoptosis. One effective approach to verify the target properties is to introduce into cancer cells specific gene-inactivating agents, such as a small interfering RNA (siRNA), that mimic antagonists and then assess cell transformation by assaying for cancer-related attributes.

However, these in vitro studies, although readily performed, can be insufficient alone to predict therapeutic value of the candidate. In vivo experiments are usually required for further validation. Human tumor xenografts in an immuno-compromised mouse (e.g. an athymic nu/nu or severe combined immunodeficiency (SCID) mouse) is the most widely accepted animal model for predicting the efficacy of treatment in patients.

However, in vivo delivery of gene inactivation agents like siRNAs is very inefficient, particularly via systemic routes. The common practice is to stably introduce siRNA vectors into cancer cells prior to transplantation, e.g. via a retroviral vector.

Such an approach can have significant drawbacks. First, the very nature of oncogenes, namely, the fact that their inactivation causes cell death or reduces growth, hinders the generation of stable knock-down cell lines and promotes the counter-selection of cells with insufficient target silencing. Second, the failure of a stable cell line to form a tumor in the animal may occur sporadically rather than due to inactivation of the target gene. Third, the phenotype does not truly mimic the human cancer therapy scenario where the clinical endpoint is regression of pre-existing tumors. For example, it may be that tumor establishment would be easier to perturb than the growth and maintenance of established tumors.

The present invention addresses this and other problems.

### BRIEF SUMMARY OF THE INVENTION

The present invention provides methods of selectively inducing siRNA expression in a cell. In some embodiments, the methods comprise the steps of:

i. transforming a cell with a multigene expression cassette comprising:

(a) a first expression cassette comprising an siRNA coding region operably linked to one or more inducible pol III promoters, and

(b) a bicistronic expression cassette having a polynucleotide encoding a detectable marker and a polynucleotide encoding a repressor, wherein the repressor represses the activity of the inducible pol III promoter; and wherein a constitutive promoter is operably linked to the detectable marker polynucleotide and the repressor polynucleotide, wherein the detectable marker polynucleotide is linked downstream of the repressor polynucleotide via an internal ribosome entry site (IRES), thereby allowing for transcription of a polycistronic RNA encoding the repressor and the detectable marker; and

ii. inducing expression of the siRNA by blocking or reducing the binding of the repressor to the inducible pol III promoter.

In some embodiments, the methods comprise culturing the cell under conditions permitting stable integration of the multigene expression cassette prior to the induction step.

In some embodiments, the methods further comprise a step of introducing the cells into a host animal prior to the inducing step. In some embodiments, the introducing step comprises implanting the cells into a xenographic host or a syngenic host. In some embodiments, the xenographic host is a SCID or athymic nu/nu mouse.

In some embodiments, the methods comprise introducing a retroviral vector into the cell, wherein the retroviral vector comprises the multigene expression cassette.

The present invention also provides integrating multigene expression cassettes comprising:

(a) a first expression cassette comprising an siRNA coding region operably linked to one or more inducible pol III promoters, and

(b) a bicistronic expression cassette having a polynucleotide encoding a detectable marker and a polynucleotide encoding a repressor, wherein the repressor represses the activity of the inducible pol III promoter; and wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

The present invention also provides libraries of cells containing a multigene expression cassette, wherein the integrating multigene expression cassette comprises:

(a) a first expression cassette comprising an siRNA coding region operably linked to one or more inducible pol III promoters, and
(b) a bicistronic expression cassette having a polynucleotide encoding a detectable marker and a polynucleotide encoding a repressor, wherein the repressor represses the activity of the inducible pol III promoter; and wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

The present invention also provides cells transformed with a multigene expression cassette, wherein the integrating multigene expression cassette comprises:

(a) a first expression cassette comprising an siRNA coding region operably linked to one or more inducible pol III promoters, and
(b) a bicistronic expression cassette having a polynucleotide encoding a detectable marker and a polynucleotide encoding a repressor, wherein the repressor represses the activity of the inducible pol III promoter; and wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

The present invention also provides transgenic non-human animals comprising an integrated recombinant multigene expression cassette, wherein the multigene expression cassette comprises:

(a) a first expression cassette comprising an siRNA coding region operably linked to one or more inducible pol III promoters, and
(b) a bicistronic expression cassette having a polynucleotide encoding a detectable marker and a polynucleotide encoding a repressor, wherein the repressor, when present, represses the activity of the inducible pol III promoter; and wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

In some embodiments, the transgenic animal is a mouse.

In some of the above-described embodiments, the repressor is a tetracycline repressor. In some embodiments, the constitutive promoter is selected from a cytomegalovirus (CMV) promoter, an SV40 promoter, an Actin gene promoter and a GAPDH gene promoter. In some embodiments, the detectable marker gene encodes an enzyme, a fluorescent protein, or a cell surface protein. In some embodiments, the detectable marker is an antibiotic resistance marker.

In some embodiments in which the expression cassettes are introduced into a cell, the cell is a mammalian cell. In some embodiments, the cell is a plant cell. In some embodiments, the cell is a cancer cell. In some embodiments, the cell is a part of a population of cells carrying different siRNA coding regions. In some embodiments, the different siRNA coding regions comprise random sequences.

### DEFINITIONS

“siRNA” or “RNAi” refers to small interfering RNAs that are capable of causing interference and can cause post-transcriptional silencing of specific genes in cells, for example, mammalian cells (including human cells) and in the body, for example, mammalian bodies (including humans). The phenomenon of RNA interference is described and discussed in, for example, Bass, Nature 411: 428-29 (2001); Elbashir et al., Nature 411: 494-98 (2001); and Fire et al., Nature 391: 806-11 (1998); and WO 01/75164, where methods of making interfering RNA also are discussed. RNAi polynucleotides can be of any length. A “RNA duplex” refers to the structure formed by the complementary pairing between two regions of a RNA molecule. siRNA can be “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16 or 15 nucleotides in length. In some embodiments, the length of the duplex is 19-27 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 2, 3, 4, 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. In some embodiments, the siRNA polynucleotide is 30, 29, 28, 27, 26, 25, 24, 23, 22, 21, 20, 19, 18, 17, 16 or 15 nucleotides in length.

RNAi polynucleotides can be based upon the sequences and nucleic acids encoding gene products to be targeted in mammals, or alternatively, an RNAi can comprise a random sequence, for example, when a library of RNAi polynucleotides are introduced into a cell to identify genes that play a role in a phenotype of interest. Generally, when an allele is substantially silenced, it will have at least 40%, 50%, 60%, 70%, 80%, 85%, 90%, 95%, 99% or even 100% reduction expression as compared to when the siRNA is not present.

An “siRNA coding sequence” refers to DNA that is transcribed to produce an siRNA. Typically one of the two strands of the resulting siRNA encodes a portion of a polypeptide or, alternatively, comprises a portion of an untranslated region of an RNA. In other cases, both strands of the resulting siRNA encode a portion of a polypeptide or, alternatively, comprise a portion of an untranslated region of an RNA.

An “expression cassette” refers to a nucleic acid, which when introduced into a host cell, results in transcription of one or more RNAs. An “integrating expression cassette” refers to an expression cassette which, when introduced into a host cell, becomes integrated into a chromosome of the host cell. In many embodiments, integration of the expression cassette into the chromosome will occur via “integration sequences.” For example, the LTRs of many retroviruses act as integration sequences. Other types of integration sequences such as sequences recognized by integrases or recombinases may also be used, though in these cases it is sometimes necessary to have a corresponding integration sequence in the target genome.

A “bicistronic expression cassette” refers to an expression cassette in which two or more cistrons are controlled by one promoter. For example, in some embodiments, a promoter is operably linked to two different open reading frames such that expression from the promoter results in transcripts comprising both open reading frames. Translation from the second open reading frame in the transcript can occur via an internal ribosome entry site, which allows for initiation of translation of the second open reading frame.

“Stable integration” refers to integration of a polynucleotide into the genome (i.e., chromosome) of a cell. In one embodiment of the invention, a viral vector that integrates into the host cell genome, such as a retroviral vector or an adenoassociated viral (AAV) vector is employed. Examples of retroviruses, from which viral vectors of the invention can be derived, include human immunodeficiency virus (HIV, a lentiviral vector), avian retroviruses such as avian erythroblastosis virus (AEV), avian leukosis virus (ALV), avian myeloblastosis virus (AMV), avian sarcoma virus (ASV), spleen necrosis virus (SNV), and Rous sarcoma virus (RSV); non-avian retroviruses such as bovine leukemia virus (BLV); feline retroviruses such as feline leukemia virus (FeLV) or feline sarcoma virus (FeSV); murine retroviruses such as murine leukemia virus (MuLV), mouse mammary tumor virus (MMTV), murine sarcoma virus (MSV), and Moloney murine sarcoma virus (MoMSV); rat sarcoma virus (RsSV); and primate retroviruses such as human T-cell lymphotropic viruses 1 and 2 (HTLV-1, 2) and simian sarcoma virus (SSV). Many other suitable retroviruses are know to those of skill in the art. Often the viruses are replication deficient, i.e., capable of integration into the host genome but not capable of replication to provide infective virus.

A “promoter” is defined as an array of nucleic acid control sequences that direct transcription of a nucleic acid. As used herein, a promoter includes necessary nucleic acid sequences near the start site of transcription, such as, in the case of a pol III I promoter, a TATA element. A promoter also optionally includes distal enhancer or repressor elements, which can be located as much as several thousand base pairs from the start site of transcription. A “constitutive” promoter is a promoter that is active under most environmental and developmental conditions. “Inducible” means that a promoter sequence, and hence the nucleic acid sequence whose expression it controls, is subject to regulation in response to factors that act as “inducers”. These factors can be proteins, nucleic acids, small molecules or physical stimuli, e.g., UV irradiation. Induction of regulated nucleic acid sequences may involve the binding of factors that directly stimulate activity, or alternatively, may require the removal of factors so as to de-repress expression of a nucleic acid sequence. Induction can be measured, for example by treating cells with a potential inducer and comparing the expression of a nucleic acid sequence in the induced cells to the activity of the same nucleic acid sequence in control samples not treated with the inducer. Control samples (untreated with inducers) are assigned a relative activity value of 100%. Induction of a nucleic acid sequence is achieved when the activity value relative to the control (untreated with inducers) is 110%, more optionally 150%, more optionally 200-500% (i.e., two to five fold higher relative to the control), more optionally 1000-3000% higher.

The term “operably linked” refers to a functional linkage between a nucleic acid expression control sequence (such as a promoter, or other array of transcription factor binding sites) and a second nucleic acid sequence, wherein the expression control sequence directs transcription of the nucleic acid corresponding to the second sequence.

A “pol III promoter” refers to a nucleotide sequence to which RNA Polymerase III can bind. Exemplary promoters include promoters of U6 snRNA, tRNAs, 5S rRNA, and H1 RNA. The pol III promoter can be from a human, mouse, rat, Drosophila or other species.

A “detectable marker” refers to a transcript or polypeptide that can be detected to determine expression levels from a promoter. Detectable markers include selectable markers, i.e., a marker which allows a cell to survive in the presence of an otherwise toxic substance. Examples of selectable markers include, e.g., antibiotic resistance genes. Detectable markers also include markers that allow one to distinguish between cells comprising the marker and those not comprising the marker, and optionally quantify expression of the marker. An example of such detectable markers includes visually detectable markers such as luciferase or green fluorescent protein.

A “repressor” refers to a protein that prevents expression from a promoter. Typically, the repressor binds to a polynucleotide sequence in or near the promoter (e.g., at a site referred to as an the “operator”) thereby preventing transcription downstream of the promoter. An example of a repressor is the tetracycline repressor (TetR), which represses transcription of tetracycline responsive promoters via binding to the tet operator. Other examples of repressors include, but are not limited to, e.g., the Lac repressor and the Mar repressor (MarR), the transcriptional repressor of the multiple antibiotic resistance (mar) operon.

An “internal ribosome entry site (IRES)” refers to a cis-acting polynucleotide sequence, which when present in an RNA, mediates internal entry of the 40S ribosomal subunit upstream of a translation initiation codon in eukaryotic and viral mRNAs.

A “xenographic host” refers to an animal into which cells from a different species has been implanted. A “syngenic” host refers to an animal into which cells from an animal of the same species has been transplanted. A “transplanted” host can be a xenographic host or a syngenic host.

The term “nucleic acid” or “polynucleotide” refers to deoxyribonucleotides or ribonucleotides, analogs thereof and polymers thereof in either single- or double-stranded form. Unless specifically limited, the term encompasses nucleic acids containing known analogs of natural nucleotides which have similar binding properties as the reference nucleic acid and are metabolized in a manner similar to naturally occurring nucleotides. Examples of such analogs include, without limitation, phosphorothioates, phosphoramidates, methyl phosphonates, chiral-methyl phosphonates, 2′-O-methyl ribonucleotides, and peptide-nucleic acids (PNAs).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### BRIEF DESCRIPTION OF THE DRAWINGS

FIGS. 1A and 1B provide a diagram of exemplary multigene expression cassettes of the invention.

FIG. 1A provides an illustration representing the bicistronic and siRNA expression cassettes of the invention.

FIG. 1B provides an illustration of an exemplary self-inactivating lentiviral expression vector derived from the human immunodeficiency virus (HIV). The U3 region of the 5′ LTR has been replaced with the CMV promoter to provide tat-independent transcription of the lentiviral genomic RNA during virus packaging. A second CMV promoter drives expression of the bicistronic expression cassette comprising TetR and an antibiotic resistance gene selected from hygro^{r}, neo^{r}, or puro^{r}. An internal ribosomal entry site (IRES) separates the TetR and antibiotic resistance gene sequences. A portion of the U3 region of the 3′ LTR has been deleted and replaced with the inducible siRNA expression cassette. The tetO-mU6 promoter is a murine U6 promoter into which the tetracycline operator (tetO) sequence has been inserted. Not shown are the HIV-1 central flap sequence and the woodchuck posttranscriptional regulatory element (WPRE) sequence located immediately upstream and downstream, respectively, of the bistronic expression cassette. The entire region between the modified LTRs has been inserted into a pBluescript vector backbone

FIG. 2 illustrates xenograft tumor models for cancer target in vivo validation.

FIG. 2A illustrates a conventional xenograft experiment utilizing stable siRNA expressing cells. Two scenarios are shown: 1) oncogene inactivation causes cell death/arrest, etc. and no cells survive for in vivo testing; or 2) stable cells obtained fail to establish tumors.

FIG. 2B illustrates a new xenograft model using an inducible RNAi construct of the present invention. This allows generation of stable cells and establishment of tumors prior to gene target inactivation, which makes staged solid tumor response to gene silencing possible.

FIG. 3 illustrates how MAP3K12 silencing reduces HCT116 cell growth/survival. HCT116 cells were co-transfected with pCMV-luc and siRNA vectors against MAP3K12 (target), CNTL (negative control) and luciferase, or Bax transgene expression vector. Three days after transfection, the cells were assayed for luciferase activity. Bax was used in the assay as an additional positive control for its cytotoxic effects.

FIG. 4 illustrates apoptotic induction by MAP3K12 silencing. HCT116 and PC3M2A cells were transiently transduced with lentiviral vectors for constitutive expression of an siRNA against MAP3K12 or a control siNRA (CNTL). The cells were assayed for apoptosis induction 48 hours post-transduction using Apoptosis ELISA plus assay kit (Roche). Data represents the fold of changes as compared to control siRNA. N=6; p<0.01 for all three cell lines.

FIG. 5 illustrates the effect of mTOR silencing on cell growth. Stable HCT116 and PC3M2ACluc cells with inducible siRNA lentiviral vector against CNTL or mTOR were generated. The cells were grown in media with or without doxycycline (1 μg/ml) for four days before the cells were harvested to determine mTOR mRNA and protein levels by Real-time RT-PCR (A for HCT116 and B for PC3) and Western blotting analysis (C). For growth phenotype assays, 1000 cells of each cell type were seeded into 96-well plates containing media with or without doxycycline (1 μg/ml final concentration). The cells were passed proportionally into the new 96-well plates before reaching confluency. Cell growth was monitored by AlamarBlue™ staining every 3 days. The doxycycline effect was expressed as the ratio of the growth under induction and non-induction and normalized to control siRNA.

FIG. 6 illustrates the effect of MAP3K12 silencing on cell growth. The same conditions apply as in FIG. 5A and FIG. 5B.

FIG. 7 illustrates induction of mTOR (A) and MAP3K12 (B) siRNA silencing on early-staged xenograft tumor growth. PC3M2ACluc cells (5×10⁶) containing inducible cassettes for CNTL, mTOR, or MAP3K12 siRNAs were injected s.c. into right flank of 20-24 female athymic nude mice. For half of the animals, doxycycline (2 mg/ml) was added to drinking water on the day of injection, while no doxycycline was provided to the other half. Tumor volumes (½×length×width²) were measured twice a week after tumor establishment. The average of tumor volumes is shown.

FIG. 8 illustrates induction of mTOR (A) and MAP3K12 (B) siRNA expression on late-staged xenograft tumor growth. PC3M cells (5×10⁶) containing inducible expression cassettes for CNTL, mTOR, or MAP3K12 siRNAs were injected s.c. into right flank of 20-24 female athymic nude mice (Simonsen lab). Sixteen days after injection, the animals with desired tumor volume (>18 mm³ for CNTL (18 total), >15 mm³ for MAP3K12 (16 total), and >19 mm³ (18 total) for mTOR) were divided into two groups. One group was dosed with doxycycline (2 mg/ml); the other was not. Other conditions are the same as in FIG. 7.

### DETAILED DESCRIPTION OF THE INVENTION

### I. Introduction

The present invention provides expression constructs allowing for inducible expression of siRNA in cells. The expression constructs comprise two components.

The first component is a promoter operably linked to a bicistronic coding region encoding 1) a repressor of transcription upstream of 2) a coding sequence for a detectable marker. The coding sequences for the repressor and the detectable marker are linked via an internal ribosome entry site, thereby allowing for translation of the detectable marker.

The second component is an siRNA expression cassette comprising an inducible promoter controlled by a repressor, wherein the inducible promoter is operably linked to an siRNA coding sequence. The bicistronic expression cassette and the siRNA expression cassette are typically delivered to a cell on one polynucleotide fragment.

The bicistronic expression cassettes are designed to allow for maintenance of expression of the repressor. Cells transfected with the constructs can be selected for expression of the detectable marker, thereby ensuring expression of the repressor due to the position of the repressor in the bicistronic message. When the repressor binds to a portion (referred to as an “operator”) of the inducible promoter, expression from the promoter is blocked. Expression of the siRNA can be subsequently induced by treating the cells with an inducer molecule that binds to and inactivates the repressor, thereby allowing for controlled induction of expression of the siRNA.

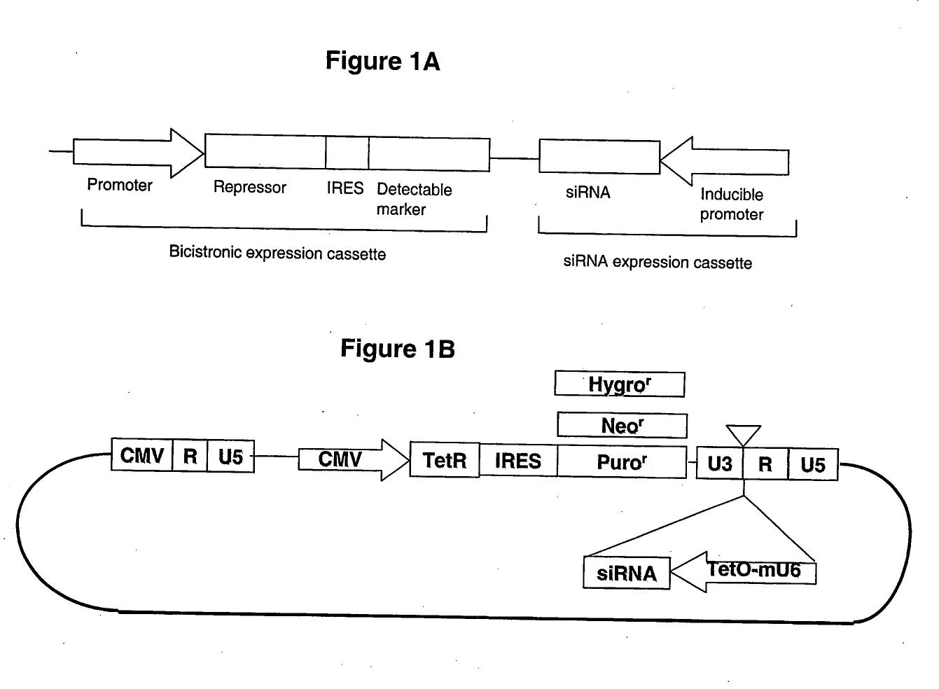

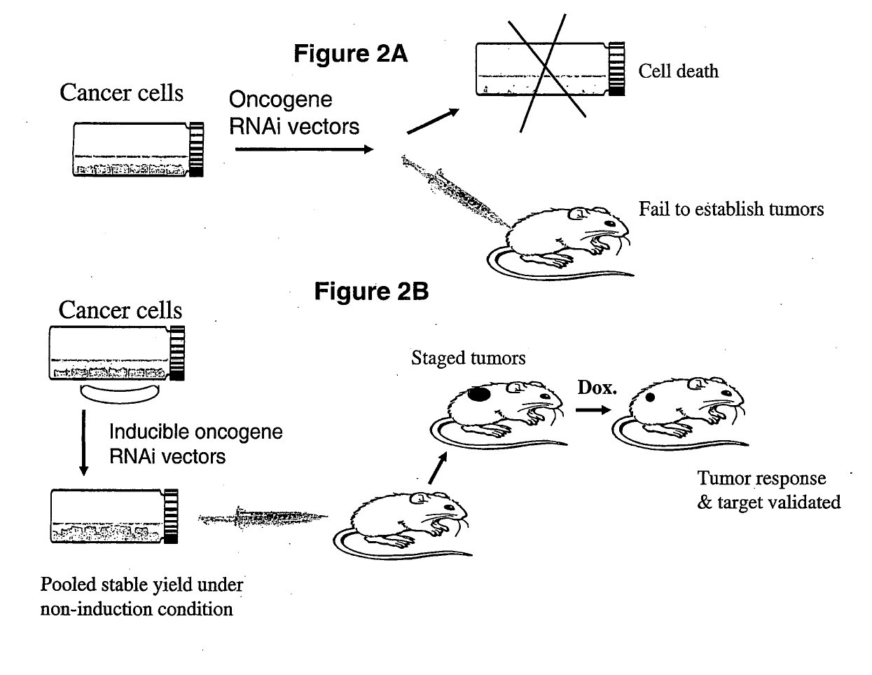

A diagram of exemplary expression constructs is displayed in FIGS. 1A and 1B.

### II. Components of the RNAi Expression Cassettes of the Invention

A. Bicistronic Promoter

The promoter for the bicistronic promoter can be any promoter that maintains expression of the repressor and detectable marker under conditions in which it is desired to repress expression of the siRNA. Generally, for ease of use, a constitutive promoter is used, though it is recognized that other promoters may also be used. Exemplary constitutive promoters for use in animal systems include, e.g., the cytomegalovirus promoter (CMV), the SV40 promoter, the Actin gene promoter, the GAPDH promoter, as well as other “house-keeping” cellular gene promoters or viral promoters.

B. Bicistron

The bicistron comprises at least two coding sequences: a repressor coding sequence and a detectable marker coding sequence. In some embodiments, the repressor is upstream of the detectable marker, thereby allowing for selection of transformants that retain and express the entire bicistron. To allow for translation of the downstream coding sequence in the bicistron (e.g., to translate the detectable marker), an internal ribosome entry sequence (IRES) is placed between the two coding sequences, thereby allowing ribosomes to translate the second coding sequence.

Any naturally or non-naturally-occurring IRES can be used. Different IRESs can be chosen depending on the cell type used. A large number of IRES sequences are known. For example, Bonnal et al., Nuc. Acids. Res. 31(1): (2003) describes a computer database which catalogs IRESs (on the internet at ifr31w3.toulouse.inserm.fr/TRESdatabase/index.htm.). See also, Helen, et al., Genes Dev. 15(13):1593-612 (2001); Vagner, et al., EMBO Rep. 2(10):893-8 (2001).

Assays to identify or test IRES sequences are known in the art and can comprise constructing a bicistronic transcript with a candidate IRES between two open reading frames. The two open reading frames generally represent two different marker genes, thereby allowing for measurement of translation of each marker. See, e.g., Crèancier, et al., J. Cell Biol., 150(1):275-281 (2000) using this technique to test the activity of a candidate IRES.

i. Repressor/Inducible Promoter/Inducer Systems

Any of a large number of repressor/inducible promoter/inducer systems may be used according to the present invention. Some of these systems involve a repressor which binds to an operator or other cis-acting sequence on an inducible promoter, thereby preventing transcription from the inducible promoter. Binding of the inducer to the repressor decreases the binding affinity of the repressor for the inducible promoter, thereby allowing the repressor to dissociate from the promoter such that expression from the promoter occurs in the presence of the inducer.

Operator sequences recognized by trans-acting factors confer inducible characteristics upon expression from promoters. Induction of expression can be accomplished by a variety of methods, depending on the particular operator system employed. For example, some operators are activated by small molecules and hormones. Exemplary operator systems include the ecdysone/glucocorticoid response element (GRE) (Invitrogen, Carlsbad, Calif.); the Tet operon (Clontech, Palo Alto, Calif.; Invitrogen, Carlsbad, Calif.); and the Lac operon (Hu and Davidson (1987) Cell, 48:555-556). Additional regulatory sequences are described, for example, in Goeddel, Gene Expression Technology: Methods in Enzymology, 185, Academic Press, San Diego, Calif. (1990). Other illustrative mammalian expression control sequences are obtained from the SV40 promoter (Science, 222:524-527 (1983)), the CMV I.E. Promoter (Proc. Natl. Acad. Sci., 81:659-663 (1984)) or the metallothionein promoter (Nature, 296:39-42 (1982)).

In some embodiments, an expression control element (operator sequence) for use with the expression cassettes of the present invention is the tetracycline (tet) operator sequence (tetO). TetO may be engineered into a modified pol III promoter, such as the U6 snRNA promoter or the H1 RNA promoter, for use with the present invention. See, e.g., U.S. Patent Publication No. 2004/0146858. When tetO is bound by a tetracycline-sensitive trans-acting protein (e.g., tetracycline repressor, TetR), transcriptional initiation at the promoter is prevented. When tetO is not bound by TetR, transcription from the promoter can proceed, allowing expression of the coding sequence operably linked to it (see, Ohkawa and Taira, Human Gene Therapy, 11:577-585 (2000); van de Wetering, EMBO Reports, 4:609-615 (2003). The inducer doxycycline (DOX), when bound to TetR, inhibits binding of TetR to tetO, thereby allowing for transcription of the downstream coding sequence. Similar results might also be achieved with tetO-modified promoters and a tetracycline transactivator protein (e.g., tTA, BD Biosciences (Clontech), Palo Alto, Calif.) instead of TetR.

ii. Detectable Markers

It is frequently desirable to have a method for identifying cells that have successfully incorporated a nucleic acid construct of the present invention. This can be accomplished through the inclusion of a detectable marker gene into the vector used in the transformation process. Detectable markers are used to distinguish cells transformed with the nucleic acid construct from those that do not. Without intending to limit the invention to particular detectable markers, detectable markers can include, e.g., markers that are visually detectable (e.g., green fluorescent protein, luciferase, etc.), enzymes that can produce detectable processed substrates (e.g., alkaline phosphatase, □-galactisidase, □-glucuronidase, etc.), cell surface proteins that can be detected using fluorophore-conjugated antibodies, and selectable markers that allow transformed cells to survive and/or thrive under conditions that harm untransformed cells (e.g., antibiotic resistance genes, such as hygromycin^{r}, neomycin^{r}, and puromycin^{r}).

C. siRNA Expression Cassette

As discussed above, an inducible promoter controlled by the repressor is operably linked to an siRNA coding region. Any inducible promoter controlled by a repressor can be used. Indeed, an operator that binds the repressor can be engineered downstream from a heterologous promoter, thereby adding the characteristic of inducibility to the promoter. In some embodiments, an operator sequence is linked to a promoter (e.g., a pol II or pol III promoter) to form an inducible promoter that is operably linked to the siRNA coding region. Examples of pol III promoters include, e.g., promoters for U6 snRNA, tRNAs, 5S rRNA, and H1 RNA.

Optionally, a second inducible promoter can be linked downstream in the opposite orientation from the first inducible promoter to form an siRNA expression cassette that expresses a double stranded siRNA. See, e.g., PCT Publication No. WO/2004/009794. The first and second inducible promoters can be identical, but to avoid recombination and vector instability, the two promoters preferably are different promoters. Typically, however, the same operator will be present in both inducible promoters to allow for induction of both promoters equally. However, it is understood that different operators could also be used in a system involving two different inducers.

The siRNA expression cassette can flank either side of the bicistronic expression cassette. For embodiments in which the expression cassettes are inserted into a retroviral vector, it can be beneficial, though not necessary, to insert the siRNA expression cassette into or adjacent to the U3 region of the 3′ LTR of the retroviral vector. The 3′ LTR is duplicated during reverse transcription (see, e.g., Field's Virology, Fourth Ed., Vol. 2 (Eds., Knipe & Howley, 2001)), therefore, the provirus acquires two copies of the siRNA expression cassette. See, e.g., Tiscornia et al., Proc. Natl. Acad. Sci. USA, 100: 1844-1848 (2003). An additional benefit of the insertion of the siRNA expression cassette into or adjacent the U3 region of non-self-inactivating retroviral vectors is that it may disrupt the pol II promoter activity of the 5′ LTR. The pol II promoter activity of the 5′ LTR may have a negative effect (promoter interference) on the expression of the other cassettes in the vector (e.g., the CMV promoter). Therefore, disruption of the pol II promoter activity of the 5′ LTR by the siRNA expression cassette may minimize this effect. However, disruption of the LTR promoter activity is already achieved in self-inactivating retroviral vectors. See, e.g., Miyoshi et al., J. Virol., 72: 8150-8157 (1998).

The siRNA coding sequence can be a known sequence or can be a random sequence, e.g., as a part of an siRNA random library. In some embodiments, it is desirable to confirm or test the effect of suppression of a particular gene product in a cell. As discussed in more detail below, such characteristics can be cell-based or can be determined in, e.g. transgenic animals or animals in which cells have been transplanted.

Another application of the present invention is the construction of a library of expression cassettes which may be used for expressing randomized siRNAs, e.g., for identifying unknown cellular genes whose silencing by an siRNA produces a detectable change in a phenotypic character of the cell system in which the siRNA gene library is expressed.

In general terms, this method involves transfecting or transducing a population of cells with a randomized siRNA expression library. One or more biological activities of the population of cells is then monitored before and/or after induction of the siRNA. Cells showing a change in the monitored activity are isolated, and the expression cassettes containing the operative siRNA of interest selected. The siRNA of these cassettes can be expanded for subsequent rounds of screening. The sequence of the selected siRNAs from the first and/or subsequent rounds of screening can be determined, and this data is then used for searching nucleic acid databases and/or for generating probes to identify the target nucleic acid(s) associated with the alteration of the monitored character, or for use in other applications.

Construction of an siRNA gene library in accordance with the present invention can involve the synthesis of nucleic acid sequences coding for siRNAs. The nucleic acid sequences can be known or random. When the sequence is random, a family of randomized sequences can be obtained comprising (theoretically) all base permutations possible for the randomized sequence length, from a single batch synthesis. In general, this means that 4^{N} different library members will be produced, where N=the number of nucleotides in each of the randomized sequences. The members of the library can then be cloned into a bacterial vector for amplification, or can be PCR amplified using techniques well known in the art. Sambrook et al., Molecular Cloning—A Laboratory Manual (2nd ed.) Vols. 1-3, Cold Spring Harbor Laboratory, Cold Spring Harbor Press, N.Y., (Sambrook) (1989); and F. M. Ausubel et al., (eds.) Current Protocols in Molecular Biology, Current Protocols, a joint venture between Greene Publishing Associates, Inc. (1994) and John Wiley & Sons, Inc. (1994 Supplement) (Ausubel).

Each randomized nucleic acid sequence is then ligated into an expression cassette of the invention under control of the inducible promoter as described herein. Once the nucleic acid sequence is positioned in the expression cassette or expression vector, its complementary strand is synthesized. This can be done enzymatically using the Klenow fragment of E. coli DNA polymerase I, or alternatively, the expression cassette can be incorporated into a vector that is then used to transform a competent cell line, with the missing complementary sequence being incorporated into the expression cassette by the cells' repair enzymes.

### III. Expression Vectors

The expression cassettes of the invention can be introduced into cells by any methods known in the art. In some embodiments, the expression cassettes are introduced via recombinant vectors. Any vector capable of accepting a DNA expression cassette of the present invention is contemplated as a suitable recombinant vector for the purposes of the invention. The vector may be any circular or linear length of DNA that either integrates into the host genome or is maintained in episomal form. Vectors may require additional manipulation or particular conditions to be efficiently incorporated into a host cell (e.g., many expression plasmids), or can be part of a self-integrating, cell specific system (e.g., a recombinant virus).

Each vector system has advantages and disadvantages, which relate, among others, to host cell range, intracellular location, level and duration of dsRNA expression, and ease of scale-up/purification. Choice of vector may also depend on the intended application.

Vector systems useful for the present invention include viral vectors, e.g., retroviruses, lentiviruses, adenoviruses, adeno-associated viruses, baculovirus, etc. Exemplary mammalian viral vector systems include replication defective retroviral vectors, lentiviral vectors, adenoviral vectors, adeno-associated type 1 (“AAV-1”) or adeno-associated type 2 (“AAV-2”) vectors, hepatitis delta vectors, live, attenuated delta viruses and herpes viral vectors.

Retroviruses are RNA viruses that are useful for stably incorporating genetic information into the host cell genome. When a retrovirus infects a cell, its RNA genome is converted to a dsDNA form (by the viral enzyme reverse transcriptase). The proviral DNA is efficiently integrated into the host genome, where it permanently resides, replicating along with the host DNA at each cell division. The integrated provirus steadily produces viral RNA from a strong promoter located at the 5′ end of the genome (in a sequence called the long terminal repeat or LTR). This viral RNA serves both as mRNA for the production of viral proteins and as genomic RNA for new viruses. Viruses are assembled in the cytoplasm and bud from the cell membrane, usually with little effect on the cell's health. Thus, the retrovirus genome becomes a permanent part of the host cell genome, and any foreign gene placed in a retrovirus ought to be expressed in the cells indefinitely. Retroviruses are therefore attractive vectors because they can permanently express a foreign gene in cells. Most or possibly all regions of the host genome are accessible to retroviral integration (Withers-Ward et al., Genes Dev., 8:1473-1487 (1994)).

Retroviral vector particles are prepared by recombinantly inserting an expression cassette of the present invention into a retroviral vector and packaging the vector with retroviral proteins by use of a packaging cell line or by co-transfecting non-packaging cell lines with the retroviral vector and additional vectors that express retroviral proteins. The resultant retroviral vector particle is generally incapable of replication in the host cell but integrates into the host cell genome as a proviral sequence containing the expression cassette containing a nucleic acid encoding a dsRNA. As a result, the host cell produces the dsRNA encoded by the nucleic acid of the expression cassette.

Packaging cell lines are generally used to prepare the retroviral vector particles. A packaging cell line is a genetically constructed mammalian tissue culture cell line that produces the necessary viral structural proteins required for packaging, but which is incapable of producing infectious virions. Retroviral vectors, on the other hand, lack the structural genes but have the nucleic acid sequences necessary for packaging. To prepare a packaging cell line, an infectious clone of a desired retrovirus, in which the packaging site has been deleted, is constructed. Cells transformed with this construct will express all structural proteins but the introduced DNA will be incapable of being packaged. Alternatively, packaging cell lines can be produced by introducing into a cell line one or more expression plasmids encoding the appropriate core and envelope proteins. In these cells, the gag, pol, and env genes can be derived from the same or different retroviruses.

A number of packaging cell lines suitable for the present invention are available in the prior art. Examples of these cell lines include Crip, GPE86, PA317 and PG13. See Miller et al., J. Virol., 65:2220-2224 (1991). Examples of other packaging cell lines are described in Cone and Mulligan, Proceedings of the National Academy of Sciences, U.S.A., 81:6349-6353 (1984) and in Danos and Mulligan, Proceedings of the National Academy of Sciences, U.S.A., 85:6460-6464 (1988); Eglitis et al., Biotechniques, 6:608-614 (1988); Miller et al., Biotechniques, 7:981-990 (1989). Amphotropic or xenotropic envelope proteins, such as those produced by PA317 and GPX packaging cell lines may also be used to package the retroviral vectors.

Defective retroviruses are well characterized for use in gene transfer to mammalian cells (for a review see Miller, A. D., Blood, 76:271 (1990)). A recombinant retrovirus can be constructed having a nucleic acid encoding an expression cassette of the present invention inserted into the retroviral genome. Additionally, portions of the retroviral genome can be removed to render the retrovirus replication defective. The replication defective retrovirus is then packaged into virions that can be used to infect a target cell through the use of a helper virus by standard techniques.

Adenoviruses can also be used to deliver the expression cassettes of the invention. The genome of an adenovirus can be manipulated such that it encodes an expression cassette of the present invention, but is inactivated in terms of its ability to replicate in a normal lytic viral life cycle. See, for example Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 d1324 or other strains of adenovirus (e.g., Adz, Ad3, Ad7 etc.) are well known to those skilled in the art.

Adeno-Associated Viruses (AAV) can also be used to deliver the expression cassettes of the invention. Adeno-associated virus (AAV) is a naturally occurring defective virus that requires another virus, such as an adenovirus or a herpes virus, as a helper virus for efficient replication and a productive life cycle. (For a review see Muzyczka et al., Curr. Topics in Micro. and Immunol., 158:97-129 (1992)).

The expression cassettes of the present invention may also be incorporated into lentiviral vectors. In this regard, see, e.g., Qin et al. (2003) Proc. Natl. Acad. Sci. USA 100: 183-188; Miyoshi et al. (1998) J. Virol. 72: 8150-8157; Tisconia et al. (2003)Proc. Natl. Acad. Sci. USA 100: 1844-1848; and Pfeifer et al. (2002) Proc. Natl. Acad. Sci. USA 99: 2140-2145. Lentiviral vector kits are available from Invitrogen (Carlsbad, Calif.).

### IV. Integration Sequences

Integration sequences can be included in the expression cassettes of the invention to allow for ease of stable integration of the expression cassettes into the genome of a host cell. As discussed above, when using retroviral vectors that integrate into the genome, integration sequences are generally included in the LTR sequence.

Alternatively, other integration sites can flank the expression cassettes of the invention. For example, sequences recognized by an integrase or recombinase can be used to assist integration and recombination of a polynucleotide into the genome of a host cell. Exemplary integration sites include, e.g., lox sequences, which are recognized by the Cre enzyme. lox sites include, but are not limited to, LoxB, LoxL, LoxC2 and LoxR sites, which are nucleotide sequences isolated from E. coli (see, e.g., Hoess et al. (1982) Proc. Natl. Acad. Sci. U.S.A. 79:3398). Lox sites can also be produced by a variety of synthetic techniques (see, e.g., Ito et al. (1982) Nuc. Acid Res. 10:1755 and Ogilvie et al. (1981) Science 270:270). Integration sites can also include, but are not limited to, those recognized by the int/att system of lambda phage, the FLP/FRT system of yeast, the Gin/gix recombinase system of phage Mu, the Cin recombinase system, the Pin recombinase system of E. coli and the R/RS system of the pSR1 plasmid.

### V. Host Cells

The expression cassettes of the present invention can be used to transform any eukaryotic or prokaryotic cell for a variety of purposes including, but not limited to, amplification of the expression cassette sequence, reverse genomic studies and gene therapy. Eukaryotic cell types that can serve as targets for vectors containing expression cassettes of the present invention include primary cell cultures, cell lines, yeast, and cellular populations in whole organs and organisms.

The invention is not limited to the type of organism or type of cell in which RNA is expressed. Any organism in which the function of a DNA sequence is sought to be determined or in which expression of a DNA sequence is to be silenced in response to treatment with an inducer is contemplated to be within the scope of the invention. Such organisms include, but are not restricted to, animals (e.g., vertebrates, invertebrates.), plants (e.g., monocotyledon, dicotyledon, vascular, non-vascular, seedless, seed plants), protists (e.g., algae, citliates, diatoms), and fungi (including multicellular forms and the single-celled yeasts).

In addition, any type of cell into which an expression vector may be introduced is expressly included within the scope of this invention. Such cells are exemplified by embryonic cells (e.g., oocytes, sperm cells, embryonic stem cells, 2-cell embryos, protocorm-like body cells, callous cells), adult cells (e.g., brain cells, fruit cells), undifferentiated cells (e.g., fetal cells, tumor cells), differentiated cells (e.g., skin cells, liver cells), dividing cells, senescing cells, cultured cells, and the like.

Eukaryotic host cells for use in the disclosed method include, but are not limited to, monkey kidney CVI line transformed by SV40 (COS-7, ATCC CRL 1651); human embryonic kidney line (293, Graham et al., J. Gen Virol., 36:59 (1977)); baby hamster kidney cells (BHK, ATCC CCL 10); Chinese hamster ovary-cells-DHFR(CHO, Urlaub and Chasin, Proc. Natl. Acad. Sci. (USA), 77:4216 (1980)); mouse sertoli cells (TM4, Mather, Biol. Reprod., 23:243-251 (1980)); monkey kidney cells (CVI ATCC CCL 70); African green monkey kidney cells (VERO-76, ATCC CRL-1587); human cervical carcinoma cells HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, BB 8065); mouse mammary tumor (MMT 060562, ATCC CCL51); TRI cells (Mather et al., Annals N.Y. Acad. Sci, 383:44-68 (1982)); human B cells (Daudi, ATCC CCL 213); human T cells (MOLT-4, ATCC CRL 1582); and human macrophage cells (U-937, ATCC CRL 1593). The cells can be maintained according to standard methods well known to those of skill in the art (see, e.g., Freshney, Culture of Animal Cells, A Manual of Basic Technique, (3d ed.) Wiley-Liss, N.Y. (1994); Kuchler et al., Biochemical Methods in Cell Culture and Virology (1977), Kuchler, R. J., Dowden, Hutchinson and Ross, Inc. and the references cited therein). Cultured cell systems can be in the form of monolayers of cells or cell suspensions.

### VI. Introduction of Expression Cassettes into Animals

The expression cassettes of the invention can be introduced into animals in several different ways. For example, in some embodiments, the expression cassettes can be introduced into cells and the cells can be subsequently introduced into an animal. The introduced cells can be those from the animal to which the cells are implanted, or can be from a different animal of the same species or of a different species (i.e., a “xenograft”). Generally, animals used for xenografts have significantly reduced immune responses, thereby allowing for introduction and maintenance of foreign cells in the animal. Exemplary xenograft hosts include, but are not limited to, SCID mice and athymic nu/nu mice.

In some embodiments, the expression cassettes of the invention are introduced into human cells and those cells are subsequently introduced into a xenographic host. A benefit of the present invention is that the expression cassettes initially do not express the siRNA of interest. Thus the cells can be implanted and become established in the host animal prior to induction in the animal. In some embodiments, the implanted cells establish tumors prior to induction of the siRNA. These sorts of systems are useful for testing the effect of siRNA polynucleotides on cancer cell phenotypes such as uncontrolled cell growth and/or proliferation, reduced apoptosis, decreased tumor volume, etc. It is recognized that the xenograft animals comprising implanted cells of the invention can be used to test and determine the effect of siRNA polynucleotides on a wide number of diseases and disorders.

In other embodiments, non-human transgenic animals comprising the expression cassettes of the invention are produced. Transgenic animals of the invention will typically transmit the expression cassettes to their progeny, i.e., via germ cells, and therefore all the cells of the transgenic animal will contain the cassette. Transgenic animals can include, but are not limited to rodents such as mice and rats as well as rabbits, birds, primates, dogs, sheep, goats, pigs, zebrafish, nematodes, etc.

Methods of generating transgenic animals are known. One method of introducing a vector into the animal's germ line involves using embryonic stem (ES) cells or fertilized eggs as recipients of the expression vector. ES cells are pluripotent cells directly derived from the inner cell mass of blastocysts (Evans et al., Nature 292:154-156 (1981); Martin Proc. Natl. Acad. Sci. USA 78:7634-7638 (1981); Magnuson et al., J. Embryo. Exp. Morph. 81:211-217 (1982); Doetzchman et al., Dev. Biol., 127:224-227 (1988)), from inner cell masses (Tokunaga et al., Jpn. J. Anim. Reprod., 35:113-178 (1989)), from disaggregated morulae (Eistetter, Dev. Gro. Differ., 31:275-282 (1989)) or from primordial germ cells (Matsui et al., Cell 70:841-847 (1992); and Resnick et al., Nature 359:550-551 (1992)). Vectors can be introduced into ES cells using any method which is suitable for gene transfer into cells, e.g., by transfection, cell fusion, electroporation, microinjection, DNA viruses, and RNA viruses (Johnson et al., Fetal Ther., 4 (Suppl. 1):28-39 (1989)). Once the expression vector has been introduced into an ES cell, the modified ES cell is then introduced back into the embryonic environment for expression and subsequent transmission to progeny animals. The most commonly used method is the injection of several ES cells into the blastocoel cavity of intact blastocysts (Bradley et al., Nature 309:225-256 (1984)). Alternatively, a clump of ES cells may be sandwiched between two eight-cell embryos (Bradley et al., in TERATOCARCINOMAS AND EMBRYONIC STEM CELLS: A PRACTICAL APPROACH, Robertson E. J. (ed.), IRL Press, Oxford, U.K. (1987), pp. 113-151; and Nagy et al., Development 110:815-821 (1990)). Both methods result in germ line transmission at high frequency.

Transgenes may also be introduced into ES cells by, e.g., retrovirus-mediated transduction or by micro-injection. Transfected ES cells which contain the transgene may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

Transfected ES cells can thereafter colonize an embryo following their introduction into the blastocoel of a blastocyst-stage embryo and contribute to the germ line of the resulting chimeric animal. For review see Jaenisch, Science 240:1468-1474 (1988).

Alternatively, targeting vectors or transgenes may be microinjected into oocytes to generate transgenic animals. Once the expression vector has been injected into the fertilized egg cell, the cell is implanted into the uterus of a pseudopregnant female and allowed to develop into an animal. Heterozygous and homozygous animals can then be produced by interbreeding founder transgenics. This method has been successful in producing transgenic mice, sheep, pigs, rabbits and cattle (See, Jaenisch, supra; Hammer et al., J. Animal Sci., 63:269 (1986); Hammer et al., Nature 315:680-683 (1995); and Wagner et al., Theriogenology 21:29 (1984)).

Alternative methods for the production include the infection of embryos with retroviruses or with retroviral vectors. Infection of both pre- and post-implantation mouse embryos with either wild-type or recombinant retroviruses has been reported Jaenisch, Proc. Natl. Acad. Sci. USA 73:1260-1264 (1976); Jaenisch et al. Cell 24:519 (1981); Stuhlmann et al. Proc. Natl. Acad. Sci. USA 81:7151 (1984); Jahner et al. Proc. Natl. Acad. Sci. USA 82:6927-6931 (1985); Van der Putten, et al. Proc. Natl. Acad. Sci. USA 82:6148-6152 (1985); Stewart, et al. (1987) EMBO J. 6:383-388. The resulting transgenic animals are typically mosaic for the transgene since incorporation occurs only in a subset of cells which form the transgenic animal.

An alternative means for infecting embryos with retroviruses is the injection of virus or virus-producing cells into the blastocoele of mouse embryos Jahner, D. et al. Nature 298:623-628 (1982). As is the case for infection of eight cell stage embryos, most of the founders produced by injection into the blastocoele will be mosaic. The introduction of transgenes into the germline of mice has been reported using intrauterine retroviral infection of the midgestation mouse embryo Jahner, D. et al., supra. This technique suffers from a low efficiency of generation of transgenic animals and in addition produces animals which are mosaic for the transgene.

Other methods of generating transgenic animals are discussed in U.S. Pat. Nos. 6,268,545; 6,255,554; 6,222,094; 6,204,43; 6,080,912 and 5,945,577 and Great Britain patents GB2331751 and GB2318578 and in “Transgenic Animal Technology” C. A. Pinkert, ed. (1997) Acad. Press; “Gene Knockout Protocols” M. J. Tymms, et al., eds. (2001) Humana Press; and “Gene Targeting: A Practical Approach” A. L. Joyner, ed. (2000) Oxford Univ. Press.

### VII. Kits

The invention provides also kits for the practice of the methods of this invention. The kits can comprise one or more containers containing a multigene expression cassette and/or siRNA gene vector of this invention. Optionally, the kits can comprise a library of siRNA vectors. The kit can optionally include buffers, culture media, vectors, sequencing reagents, labels, antibiotics for selecting markers, and the like.

The kits may additionally include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention.

### EXAMPLES

We have developed a unique lentiviral vector, pTRIP, comprising an inducible hairpin siRNA expression cassette and a constitutive expression cassette for a bicistronic message encoding the tet repressor (TetR) as well as a selection marker. See, FIG. 1B. This “all-in-one” vector enables a single transduction to generate stable cell populations with all the necessary components for controlled gene silencing, thus avoiding pre-requirements for generating TetR cancer cell lines, via sequential transfection or co-transfection for each of the different xenografts. This approach significantly increases the robustness of the system and dramatically improves productivity. We have demonstrated that this vector can be used to generate stable cells with inducible siRNA expression by a single transduction, and without lengthy selection of single cell clones.

In the current study, we evaluated this inducible siRNA expression system for utility in an in vivo xenograft tumor model for efficacy validation of cancer targets. We assessed two oncogenes, mTOR (mammalian target of rapamycin), a well known cancer target as a positive control, and MAP3K12 (mitogen-activated protein kinase 12), a novel cancer target candidate (also referred to herein as “ZPK”). mTOR is a serine/threonine kinase that functions downstream of Akt to regulate cell growth and proliferation. The mTOR inhibitor rapamycin and its derivatives are currently under clinical evaluation for therapy of certain cancers (Bjornsti M. A. and P. J. Houghton., Nat Rev Cancer. 4:335-48 (2004)). MAP3K12, also known as ZPK, DLK, and MUK, is a member of a mixed lineage kinase family. It contains a leucine zipper domain, through which it can form heterodimers with leucine zipper transcription factors such as CREB and Myc (Reddy et al., Oncogene 18: 4447-44484 (1999)), and is involved in JNK activation pathway (Hirai, et al., Oncogene 12:641-650 (1996); Xu Z, et al., Mol Cell Biol. 21:4713-24 (2001)). MAP3K12 is also associated with transformation phenotypes in the HeLa/HF system based on our expression profiling analysis and it has also been shown to be over-expressed in certain cancers, thus suggesting a potential oncogenic role in cell transformation.

### Results:

SiRNA Targeting MAP3K12 Reduces Cancer Cell Growth/Survival

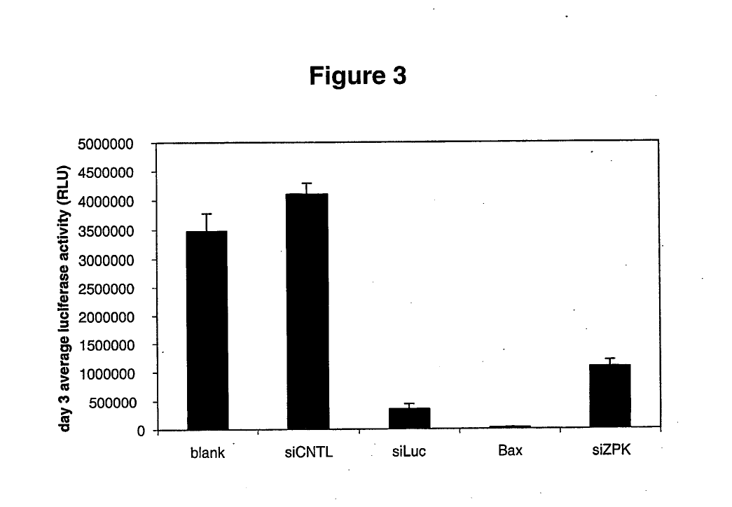

To investigate whether MAP3K12 has a pro-survival function, we investigated the effect of siRNA mediated silencing on cancer cell growth/survival. Recently we reported an effective reporter assay for cell survival/growth (Yu, et al., “A 96 well surrogate survival assay, coupled with a special RNAi vector strategy, for cancer gene target identification and validation with enhanced signal/noise ratio.” Preclinica (2005) in press). This approach involves a transient cotransfection of the luciferase reporter gene and a specific siRNA vector. The advantage of this approach is that only transfected cells generate a luciferase signal, allowing us to look only at the cytotoxicity in transfected cells, hence increasing the signal/noise ratio. The MAP3K12 siRNA vector caused significant reduction in cell survival in the HCT116 colon cancer cell line as compared with a control vector (FIG. 3), suggesting a causal effect of MAP3K12 on cancer cell survival. MAP3K12 siRNA also decreased cell survival in several other cancer cell lines tested, as shown in Table 1, using either the luciferase reporter or AlamarBlue staining and transient transfection of in vitro transcribed (IVT) MAP3K12 siRNA (Ke N., et al., BioTechnique. 36:826-833 (2004)), implying a broad pro-survival property of MAP3K12 protein.

TABLE 1
Cancer cell survival affected by MAP3K12 silencing

| CELL LINES | CANCER | | |
|---|---|---|---|
| TESTED | TYPES | Assay used | siRNA |
| HeLa | Cervical C. | AlamarBlue staining | In vitro |
| | | | transcribed |
| | | | (IVT) |
| DLD1 | Colon C. | AlamarBlue staining | IVT |
| U87 | Glioma | AlamarBlue staining | IVT |
| A549 | NSCL | Luciferase activity | Vector |
| Mcf-7 | Breast C. | Luciferase activity | Vector |
| MDA-MB231 | Breast C. | Luciferase activity | Vector |
| PC3 | Prostate C. | AlarmaBlue staining/ | Vector |
| | | Luciferase activity | |
| HCT116 | Colon C. | Luciferase activity | Hairpin vector |

SiRNA Targeting MAP3K12 Induces Apoptosis

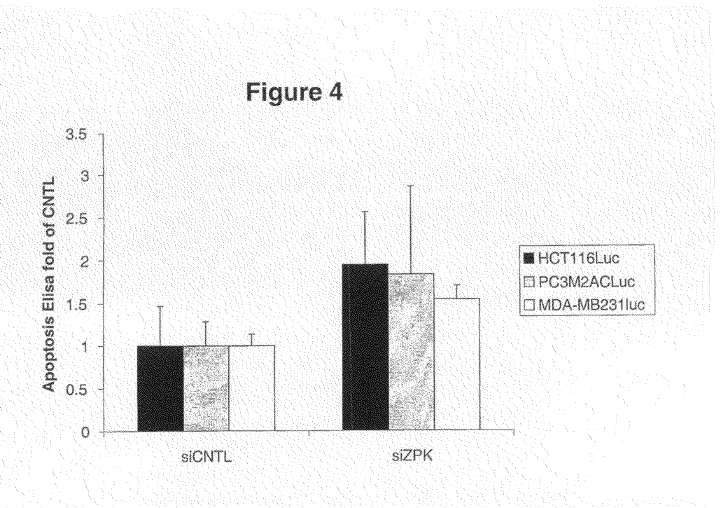

We investigated whether MAP3K12 siRNA causes apoptosis in cancer cells using the transient transduction/apoptosis assay that we developed earlier (Ke N, et al., Cancer Res. 64: 8308-12 (2004)). The advantage of this approach is that transient transduction causes little cytotoxicity as compared to transient transfection, and also avoids problems associated with counter selection that plagues stable cell transduction (Ke N, et al., Cancer Res. 64: 8308-12 (2004)). Using this approach we clearly demonstrated that MAP3K12 siRNA caused elevated apoptosis in several cancer cell lines tested, including HCT116, PC3M2ACluc and MA-MB231luc cell lines (FIG. 4), thus confirming its pro-survival functions. This result also suggests the putative oncogene properties of MAP3K12 are likely attributed to its involvement in apoptosis.

Establishment of Stable Cell Lines Containing an Inducible MAP3K12 siRNA

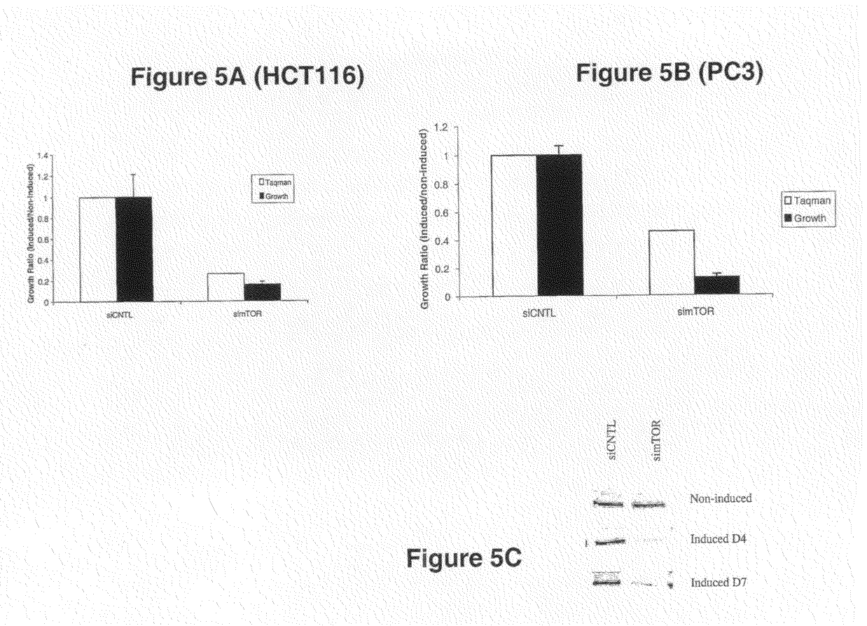

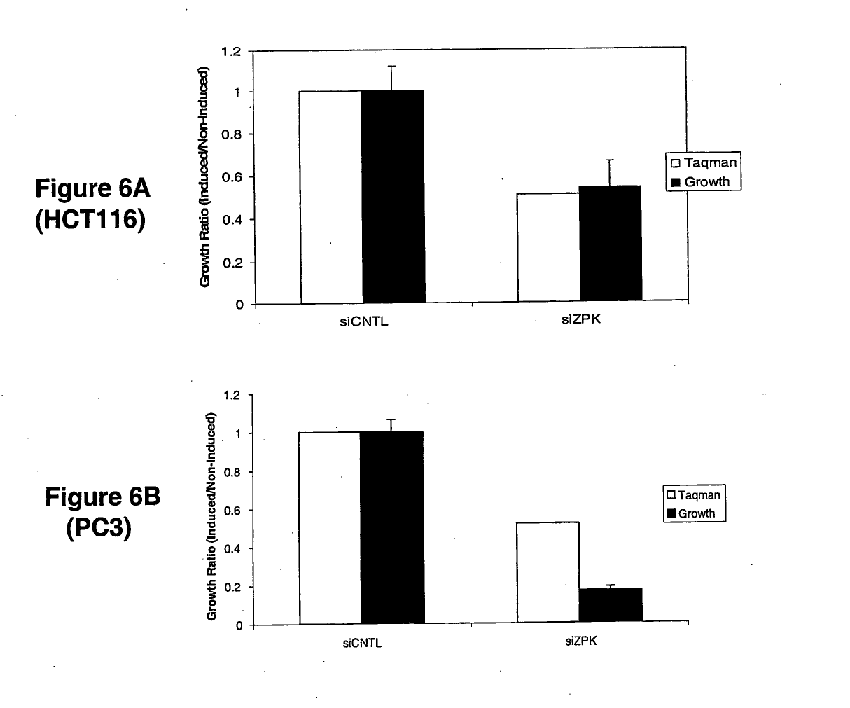

We attempted to generate cells with stable MAP3K12 down-regulation using lentiviral siRNA vectors to elucidate the mechanisms through which MAP3K12 exerts its pro-survival functions. However, multiple attempts failed in a number of cancer cell lines including PC3 (prostate cancer), HeLa (cervical cancer) and DLD1 (colon carcinoma), suggesting possible cytotoxicity caused by MAP3K12 silencing. Therefore, we developed an inducible siRNA expression system (FIG. 1B). We inserted the tetO element between the PSE and TATA elements of the mU6 promoter so that transcription is repressed in the presence of the tet repressor. SiRNA expression from the resulting tetO-mU6 promoter is induced by addition of doxycycline (DOX). We tested this vector using an siRNA against the known oncogene mTOR (Yu et al., 2005, supra). A randomized sequence was used as a negative control (CNTL) siRNA. We stably transduced the HCT116 colon carcinoma and PC3 prostate cancer cell lines with pTRIP vectors for inducible expression of mTOR, MAP3K12, or CNTL siRNAs (simTOR, siMAP3K12, or siCNTL, respectively). When pTRIP/simTOR-transduced cells were induced with DOX (1 μg/ml) for four days, both mTOR mRNA (FIG. 5A for HCT116 and 5B for PC3) and protein (FIG. 5C, >80% reduction) levels were down-regulated compared to either non-induced cells or cells transduced with the pTRIP/siCNTL vector, thus demonstrating effective induction of siRNA expression. As expected, we observed a sharp decline of growth/survival under induced conditions for cells expressing the mTOR siRNA beginning on day 4 and becoming more pronounced by day 9 as assessed by alamarBlue staining (FIG. 5A for HCT116 and 5B for PC3). This is consistent with the known oncogenic properties of mTOR. Likewise, MAP3K12 mRNA was down-regulated upon induction of cells transduced with the pTRIP/siMAP3K12 vector. Cell growth/survival of these cells was also greatly reduced as measured by alamarBlue staining (FIG. 6A for HCT116 and 6B for PC3). These results further demonstrate the oncogenic nature of MAP3K12.

Validation of MAP3K12 in an Inducible RNAi Xenograft Model

We then evaluated mTOR and MAP3K12 as cancer targets using cells stably transduced with the pTRIP/simTOR of pTRIP/siMAP3K12 vectors in the xenograft model. Two variations of this model were used to assess comprehensively these two targets. “Early staged tumors” refers to tumors implanted only a few days before the treatments became effective. This usually occurs before a tumor is detectable, and is therefore used for xenograft tumors with a 90-100% take-rate. “Later staged tumors” with measurable tumor sizes closely mimic real human cancers and can be used for tumor regression response analysis. Subcutaneous tumor transplantion was used to facilitate easy measurement of tumor volumes and thus tumor response. Induction of gene silencing was achieved by continuous oral dosing via drinking water containing 0.5-2 mg/ml of DOX.

The PC3 prostate cancer cell line was used because of its near 100% take-rate in the athymic mouse, thus enabling use of the “early staged” tumor model. Stably transduced PC3 cells were first tested by implanting into nude mice (nu/nu) subcutaneously (s.c.) at the right flank of the animals (5×10⁶ cells per mouse, 12×3 mice with mTOR, 12×2 mice for MAP3K12 siRNA vectors, and 10×2 mice with control vector). The detailed schedules of the treatment and the number of animal groups are summarized in Table 2. Two induction schedules were applied in the experiments: 1) continuous dosing starting on Day 0 after implantation, allowing tiny tumors (early staged) to establish before the target silencing, since our induction kinetics in vitro indicates that the mRNA knock-down was detected at least two days after induction (see above); and 2) dosing starting on day 16 post-implantation, allowing later staged tumors to form before induction. The tumor response to MAP3K12 and mTOR silencing was assessed by measuring tumor volume twice a week (Vol.=½(length×width²)).

TABLE 2
Oral DOX dosing schedule for PC3 xenograft animals.

| NO. OF | | |
|---|---|---|
| ANIMALS | Vectors | DOX (2 mg/ml)/5% sucrose |
| 10 | CNTL | No Dox |
| 11 | MAP3K12 | No Dox |
| 12 | mTOR | No Dox |
| 10 | CNTL | Dox on D0 |
| 12 | MAP3K12 | Dox on D0 |
| 12 | mTOR | Dox on D0 |
| 10 | CNTL | Dox on D16 |
| 11 | MAP3K12 | Dox on D16 |
| 12 | mTOR | Dox on D16 |

In the first model, tumors continued to grow through day 7 when the majority of tumor sizes are measurable, and regression was first observed on day 10. By day 17, regression, as compared to day 7 tumor volume, was dramatic for both mTOR and ZPK siRNAs: 100% of tumors (12/12) regressed and 50% (6/12) of the animals became tumor-free (FIG. 6). Most tumor-free animals (6/6 for mTOR siRNA and 4/6 for ZPK siRNA) remained so while DOX was provided (through day 30).

In the second model, tumors were allowed to form measurable sizes (near 30 mm³ in volume) and were at their rapid growth phase under non-induced conditions. On day 16, non-induced animals implanted with pTRIP/siCNTL, pTRIP/simTOR-, or pTRIP/siMAP3K12-transduced PC3 cells were divided into two subgroups based on their respective tumor volumes (>18 mm³, 18/20 for siCNTL; >15 mm³ 16/22 for siZPK; >19 mm³ 18/24 for simTOR). The two subgroups were divided so that the average tumor volumes (>20 mm³ and <20 mm³) were similar among them. One group was then treated with DOX and another was not. It is worth noting that DOX itself has a slight effect on the growth of tumors with CNTL siRNA, consistent with a previous report that DOX inhibits cancer cell growth and metastasis (Saikali Z, Singh G. Anticancer Drugs. 14: 773-8 (2003)). However, none of the tumors regressed in all nine animals with CNTL siRNA. Since we subsequently found that the same mRNA knock-down was achievable with much lower doses of DOX, we may be able to minimize the non-specific inhibitory effect on tumor growth in future experiments by reducing the dosage of DOX.

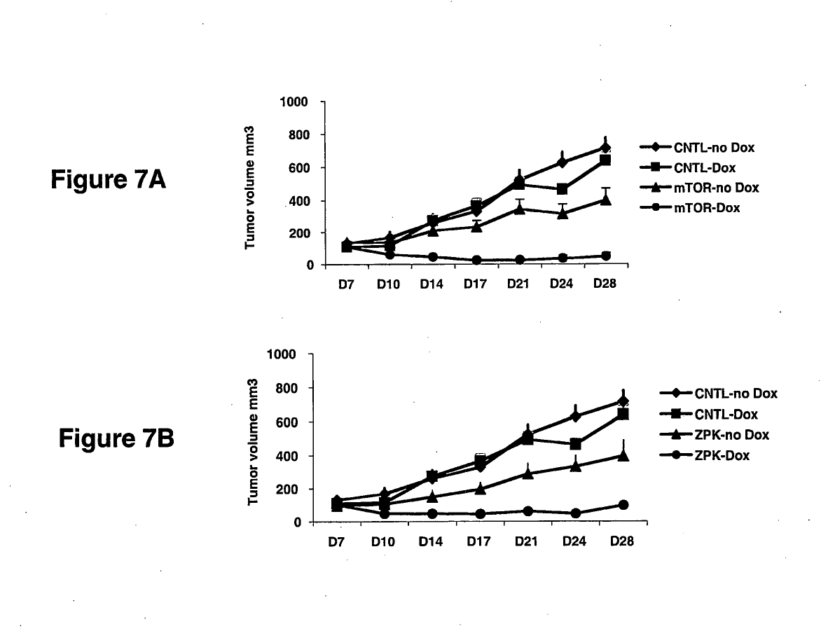

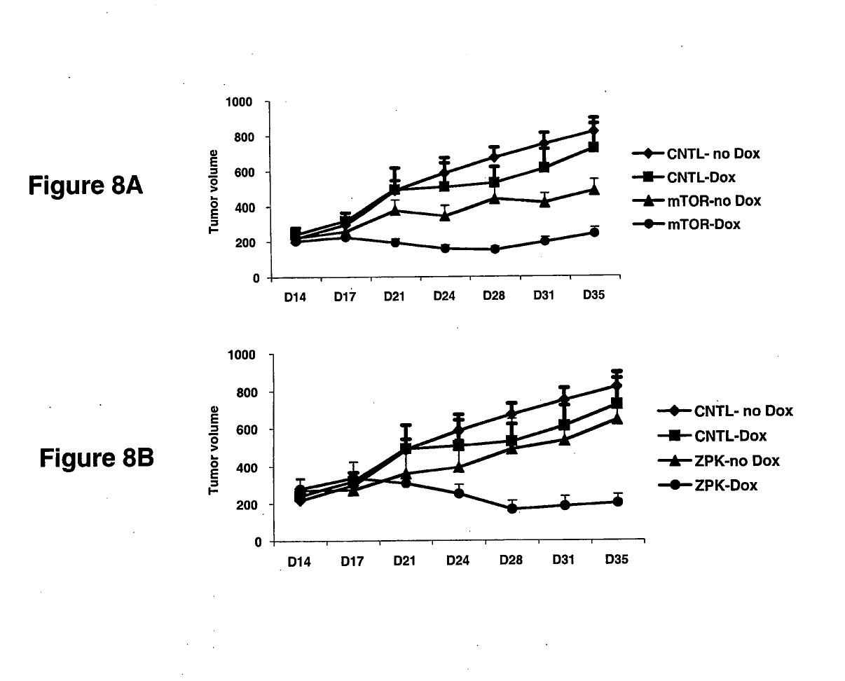

The DOX effect on tumors expressing mTOR or MAP3K12 siRNAs was much more pronounced. In these animals, the tumors stopped growing and started to regress slowly but convincingly 7 days after induction in contrast to the non-induced animals in which the tumors continued to grow (FIG. 7). For mTOR siRNA, 100% (10/10) of the tumors regressed; for MAP3K12 siRNA, 75% (6/8) of the tumors regressed, although none of them became tumor-free. The remaining 25% (2/8) of tumors with MAP3K12 siRNA displayed either arrested or attenuated growth relative to controls (CNTL vector+/−DOX, and mTOR/MAP3K12 siRNA vector without DOX). The regressed tumors did not grow again until day 31 when DOX was removed, and even then at a slower rate. Overall, as compared to controls (CNTL vector+/−DOX, and mTOR/MAP3K12 siRNA vector without DOX), all the mTOR silenced tumors (100% or 9/9) and MAP3K12 silenced tumors (87% or 7/8) regressed in response to the induction, further validating these genes as potential drug targets for cancer treatment.

### Discussion

Targeted medicine is considered the future of cancer therapy. Gleevec (Novartis, AG), Iressa (AstraZenaca) and Erbitux (ImClone, Inc.) are successful examples of targeted therapy for cancers, demonstrating significantly improved efficacy and lower toxicity than conventional chemotherapy drugs. Functional genomic technologies have greatly facilitated identification of a large number of candidate cancer targets. Although most of these targets have been or can be validated in vitro by phenotypic assays based on transgene expression (gain of function) or gene inactivation (loss of function), very few have been further validated in vivo due to lack of effective tools and high cost. Validation using effective animal models has been a major bottleneck for drug discovery in oncology. Xenograft tumor formation has been the most widely used animal model to evaluate efficacy because it provides one of the best predictors for human cancer treatment. However, few tools are available to take full advantage of this model for target validation. This report is the first to demonstrate an inducible knock-down xenograft model which measures tumor regression in direct response to gene target inactivation. We believe this approach will become an essential tool for cancer target validation.

Several factors are crucial to the success and broad application of this model, including tight regulation of siRNA expression and the choice of the siRNA itself. A number of TetR-based inducible siRNA vectors have been described (Chen Y, et al., Cancer Res. 63:48014 (2003), Matsukura S, et al., Nucleic Acids Res. 31:e77 (2003), Wiznerowicz M, Trono D. J. Virol. 77:8957-61 (2003)). However, these required co-transfection or co-transduction of the cells with a TetR expression vector and a separate vector carrying the inducible siRNA cassette. In addition, some of them required single cell cloning to generate stable, inducible siRNA cell lines. In our hand, these vectors were not universally reliable for all targets and/or cell models, primarily due to the leaky nature of the siRNA expression cassette. Therefore, we developed this “all-in-one” lentiviral vector for inducible siRNA expression that also constitutively expresses high levels of TetR, thus avoiding multiple transduction/transfection steps and single cell cloning. The time required for stable line generation is thus significantly reduced. This vector exhibits minimal silencing of the target gene under non-induced conditions (similar to constitutive expression of control siRNAs) and substantial silencing of the target gene under induced conditions (similar to constitutive expression of target siRNAs. These properties expand the threshold window for adequate phenotype induction. Furthermore, our preliminary observations indicate that it could be possible to regulate the levels of gene silencing by varying the DOX concentration.

Our new tumor response model further supports mTOR as a validated drug target for cancer treatment. Based on preclinical and early clinical studies indicating efficacy of the current compounds targeting this gene, our results demonstrate the utility of the model for in vivo validation of novel targets.

### MATERIALS AND METHODS

### Cells

HCT116 (ATCC), PC3M2ACluc and MDAMB231luc cells are from Xenogen (Montain View, Calif.). MDAMB231 is cultured in Dulbecco's Modified Eagle's Medium (DMEM, Invitrogen, Carlsbad, Calif.), supplemented with 10% Fetal Bovine Serum (tetracycline free FBS, BD Biosciences (Clontech)), 2 mM L-Glutamine (L-Glu, Fisher Scientific), 1X Non-Essential Amino Acids (NEAA, Irvine Scientific, Santa Ana, Calif.), and 1% Sodium Pyruvate, (Invitrogen). HCT116 and PC3M2ACluc cells were maintained in RPMI 1640 (Invitrogen) supplemented with 10% FBS (tetracycline free FBS, BD Biosciences (Clontech)) and 2 mM L-Glu. Cells were maintained in a humidified incubator with 5% CO₂ at 37° C.

### Measurement of Cell Growth

Anchorage-dependent growth in 96-well was described in detail previously (Ke N, et al., Cancer Res. 64: 8308-12 (2004); Ke N., et al., BioTechnique. 36:826-833 (2004)). Cell growth in 96-well plates was scored by alamarBlue staining (Biosource International, Camarillo, Calif.). For cell growth with inducible siRNA constructs, 1000 PC3M2Acluc and HCT116luc cells with the inducible siRNA cassettes were grown in media with or without doxycycline (1 μg/ml). Cells were passaged before reaching confluency. Cell growth was monitored every 2-3 days using AlamarBlue staining.

Co-Transfection of Luciferase or lacZ Reporter Gene with siRNA and cDNA Expression Vectors

Luciferase or lacZ gene expression cassette vectors were co-transfected with expression vectors for siRNAs targeting MAP3K12 or luciferase; a non-targeting control siRNA, or Bax cDNA using TransIT-LT1 transfectian reagent (Mirus, Madison, Wis.) according to the manufacturer's instructions. Briefly, for HCT116 cells, 0.05 μg pGL3-control and 0.1 μg siRNA or cDNA expression vectors were mixed with 0.6 μl/well of TransIT-LT1 in 15 μl of Opti-MEM (Invitrogen) in 96 well plates and transfected into 3.0×10³ freshly detached cell suspensions. Three days post-transfection, luciferase activity was measured. Briefly, cells in white solid bottom 96 well plates were lysed by adding one culture-medium volume of Bright-glo reagent (Promega, Madison, Wis.) to each well. After incubation at room temperature for at least 2 minutes, the luciferase activity was measured in a Mithras LB 940 luminometer (Berthold technology, Germany). For soft agar cultures, the cells were lysed with one volume of Bright-glo for 10 minutes and the luciferase activity was measured.

### Vector Construction

Construction, preparation, and transduction of lentiviral vectors expressing siRNAs have been described previously (Ke N, et al., Cancer Res. 64: 8308-12 (2004); Ke N., et al., BioTechnique. 36:826-833 (2004); Waninger S. et al., J. Virol. 78:12829-12837 (2004)).

pHIV-7 Vector Construction

The pHIV-7-GFP (Banerjea, A., et al., Mol. Ther. 8:62-71 (2003); Yam, P. Y., et al., Mol. Ther. 5:479-484 (2002); Ke N., et al., BioTechnique. 36:826-833 (2004)) and pHIV-7 vectors were gifts from M. Li, J.-K. Yee, and J. Rossi (City of Hope, Duarte, Calif.). Construction of pHIV-7 from pHIV-7-GFP is described in Waninger S. et al., J. Virol. 78:12829-12837 (2004).

PTRIP (TetR-IRES-puro^{r}) Vector Construction)

The CMVp/TetR cassette was amplified by PCR from pcDNA6/TR (Invitrogen, Carlsbad, Calif.) using the following primers: 5′-gcggccgcTAGGGCCTCTGAGCTATTCC-3′ (SEQ ID NO: 1) and 5′-GAATTcTCTGCTTTAATAgGATCTGAAcTCCCGGGAaCCGCTGTACGCGGA-3′ (SEQ ID NO: 2). The PCR product was ligated into the Not I-EcoRI sites of pQCXIP (BD Biosciences (Clontech), Palo Alto, Calif.), just upstream of the IRES-puro^{r} cassette. The Bgl II/EcoRV DNA fragment from the resulting vector, comprising the CMV promoter-TetR-IRES-puro^{r} cassette, was then subcloned into BamH I/Sma I-digested pHIV-7. This intermediate vector was named pHIV-7-CMV p-TetR-IRES-puro^{r}. (Note that the unique BamHI site in the pHIV-7 backbone is destroyed in this ligation.) Next, a BglII site within the 3′ LTR of pHIV-7 was mutated to a BamHI site by site-directed mutagenesis using the Quik-Change Site-Directed Mutagenesis kit (Stratagene, La Jolla, Calif.) and the following primers: 5′-CCCAAAGAAGACAGGATCCGCTTTTTGCCTGTACT-3′ (SEQ ID NO: 3); and 5′-AGTACAGGCAAAAAGCGGATCCTGTCTTCTTTGGG-3′ (SEQ ID NO: 4). The resulting vector was named pHIV-7-2xBamHI. Next, we replaced the PflMI/KpnI fragment of pHIV-7-CMV p-TetR-IRES-puro^{r} with the corresponding PflMI/KpnI fragment of pHIV-7-2xBamHI to produce pTRIP. This vector has a pHIV-7 backbone and comprises the CMV promoter-driven Tet-IRES-puro^{r} cassette and a unique BamHI site withing the 3′ LTR to facilitate insertion of tetO-mU6-driven siRNA expression cassettes.

tetO-mU6-siRNA Cassette Construction

The tetO sequence was inserted into the mU6 promoter between the PSE and TATA elements by PCR using pSilencer (Ambion, Austin, Tex.) as the template and primers 5′-GGATCCGACGCCGCCATCTCTAG-3′ (SEQ ID NO: 5) and 5′-AAACAAGGCTTTTCTCCAAGGGATATTTATAactctatcaatgatagagTACTTTACAGTTA GGGTGAGT-3′ (SEQ ID NO: 6). TetO-mU6-siRNA cassettes were constructed by PCR as described in Waninger S. et al., J. Virol. 78:12829-12837 (2004) except that the tetO-mU6 promoter was used as the template. The primer sequences were as follows: universal 5′ primer, 5′-GAACTAGTGGATCCGACGCC-3′ (SEQ ID NO: 7), siRNA-specific 3′ primer, 5′-tgctGGATCCAAAAAA(siRNA sense strand sequence)TCTCTTGAA(siRNA antisense strand sequence)AAACAAGGCTTTTCTCCAAGGG-3′ (SEQ ID NO: 8).

SiRNA-specific 3′ primer sequences used in this example are: MAP3K12 (5′-tgctGGATCCAAAAAAgtcagaaacgtggcatctcTCTCTTGAAgagatgccacgtttctgacAAACAAGG CTTTTCTCCAAGGG-3′ (SEQ ID NO: 9)); mTOR (5′-tgctGGATCCAAAAAAGAGAAGAAATGGAAGAAATTCTCTTGAAATTTCTTCCATT TCTTCTCAAACAAGGCTTTTCTCCAAGGG-3′ (SEQ ID NO: 10)); CNTL, (5′-tgctGGATCCAAAAAAggcgcgctttgtaggattcgcTCTCTTGAAgcgaatcctacaaagcgcgccAAACA AGGCTTTTCTCCAAGGG-3′ (SEQ ID NO: 11)).

Ligation of tetO-mU6-siRNA Cassettes into pTRIP

BamHI-digested tetO-mU6-siRNA cassettes were ligated into BamHI-digested pTRIP. In some cases, this ligation step is facilitated by first ligating the tetO-mU6-siRNA cassette PCR product into pCR-Blunt II-TOPO (Invitrogen, Carlsbad, Calif.). The tetO-mU6-siRNA cassette is then released from the pCR-Blunt II-TOPO vector by BamHI digestion and ligated into BamHI-digested pTRIP. Clones in which the tetO-mU6 and CMV promoters face in opposite directions were identified by sequencing. A diagram of the resulting vector is provided in FIG. 1B.

VSV-G pseudotyped lentivirus was packaged using the lentiviral support kit (Invitrogen, Carlsbad, Calif.). PC3M2Acluc and HCT116luc cells stably expressing inducible siRNAs were transduced using standard methods (Tiscomia et al., Proc. Natl. Acad. Sci. USA, 100: 1844-1848 (2003)) and selected in media containing a desired concentration of puromycin.

Measurement of mTOR and MAP3K12 Expression

To determine the mRNA levels for mTOR and MAP3K12, cells were grown in either non-induced or induced conditions for four days before harvesting for real-time RT-PCR analysis as described (Ke N, et al., Cancer Res. 64: 8308-12 (2004); Ke N., et al., BioTechnique. 36:826-833 (2004)). To determine mTOR protein expression, HCT116 cells containing either the inducible CNTL or mTOR siRNA were grown in media with or without the doxycycline (1 ug/ml). Cells were harvested 4 days or 7 days post induction. Protein lysates were prepared and western blotting analysis was performed to determine the relative mTOR levels as described using mTOR polyclonal antibody from Santa Cruz (Claassen C., et al., Preclinica 2: 435-9 (2004)).

### Assay for Apoptosis

For transient transduction experiments, transduction was conducted in 96-well plates and apoptosis was assayed two days later using the DNA fragmentation-based Elisa assay (Cell Death Detection Elisa Plus, Roche Applied Science, Indianapolis, Ind.), which measures the amount of fragmented and solubilized nucleosomal DNA, a hallmark of apoptosis.

### Xenograft Tumor Formation

PC3M2Acluc cells with inducible siRNA cassettes against control, MAP3K12 and mTOR were generated as described above. 5e6 cells of each cell line were injected s.c. into 30-36 athymic nude mice. Drinking water contains 5% sucrose, either with doxycycline (2 mg/ml) (10-12 animals, no induction) or without, on day 0 (D0) (10-12 animals, induction for early stage tumor), and added on Day 16 (D16) (10-12 animals, induction for staged tumors). Tumor volume was measured and calculated twice a week starting on day 7 (D7) (½×length×width²).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of selectively inducing siRNA expression in a cell comprising the steps of:
i. transforming a cell with a multigene expression cassette comprising:
(a) a first expression cassette comprising an siRNA coding region operably linked to one or more inducible pol III promoters, and
(b) a bicistronic expression cassette having a polynucleotide encoding a detectable marker and a polynucleotide encoding a repressor,
wherein the repressor represses the activity of the inducible pol III promoter; and
wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker; and
ii. inducing expression of the siRNA by blocking or reducing the binding of the repressor to the inducible pol III promoter.

2. The method of claim 1, comprising culturing the cell under conditions permitting stable integration of the multigene expression cassette prior to the induction step.

3. The method of claim 1, wherein the repressor is a tetracycline repressor.

4. The method of claim 1, wherein the constitutive promoter is selected from a cytomegalovirus (CMV) promoter, an SV40 promoter, an Actin gene promoter and a GAPDH gene promoter.

5. The method of claim 1, wherein the cell is a mammalian cell.

6. The method of claim 1, wherein the cell is a plant cell.

7. The method of claim 1, wherein the cell is a cancer cell.

8. The method of claim 2, further comprising a step of introducing the cell into a host animal prior to the inducing step.

9. (canceled)

10. (canceled)

11. The method of claim 1, wherein the cell is a part of a population of cells carrying different siRNA coding regions.

12. The method of claim 11, wherein the different siRNA coding regions comprise random sequences.

13. The method of claim 1, wherein the detectable marker gene encodes an enzyme, a fluorescent protein, an antibiotic resistance marker, or a cell surface protein.

14. (canceled)

15. The method of claim 1, comprising introducing a retroviral vector into the cell, wherein the retroviral vector comprises the multigene expression cassette.

16. An integrating multigene expression cassette comprising:
(a) a first expression cassette comprising an siRNA coding region operably linked to one or more inducible pol III promoters, and
(b) a bicistronic expression cassette having a polynucleotide encoding a detectable marker and a polynucleotide encoding a repressor,
wherein the repressor represses the activity of the inducible pol III promoter; and
wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

17. The expression cassette of claim 16, wherein the repressor is a tetracycline repressor.

18. The expression cassette of claim 16, wherein the constitutive promoter is selected from a cytomegalovirus (CMV) promoter, an SV40 promoter, an Actin gene promoter and a GAPDH gene promoter.

19. The expression cassette of claim 16, wherein the detectable marker gene encodes an enzyme, a fluorescent protein, an antibiotic resistance marker, or a cell surface protein.

20. (canceled)

21. A library of cells containing a multigene expression cassette, wherein the integrating multigene expression cassette comprises:
(a) a first expression cassette comprising an siRNA coding region operably linked to one or more inducible pol III promoters, and
(b) a bicistronic expression cassette having a polynucleotide encoding a detectable marker and a polynucleotide encoding a repressor,
wherein the repressor represses the activity of the inducible pol III promoter; and
wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

22. A cell transformed with a multigene expression cassette, wherein the integrating multigene expression cassette comprises:
(a) a first expression cassette comprising an siRNA coding region operably linked to one or more inducible pol III promoters, and
(b) a bicistronic expression cassette having a polynucleotide encoding a detectable marker and a polynucleotide encoding a repressor,
wherein the repressor represses the activity of the inducible pol III promoter; and
wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

23. The cell of claim 22, wherein the multigene expression cassette is integrated into a chromosome of the cell.

24. The cell of claim 22, wherein the cell is a mammalian cell.

25. The cell of claim 22, wherein the cell is a plant cell.

26. (canceled)

27. The cell of claim 22, wherein the repressor is a tetracycline repressor.

28. The cell of claim 22, wherein the constitutive promoter is selected from a cytomegalovirus (CMV) promoter, an SV40 promoter, an Actin gene promoter and a GAPDH gene promoter.

29. The cell of claim 22, wherein the detectable marker gene encodes an enzyme, a fluorescent protein, an antibiotic resistance marker, or a cell surface protein.

30. (canceled)

31. A transgenic non-human animal comprising an integrated recombinant multigene expression cassette, wherein the multigene expression cassette comprises:
(a) a first expression cassette comprising an siRNA coding region operably linked to one or more inducible pol III promoters, and
(b) a bicistronic expression cassette having a polynucleotide encoding a detectable marker and a polynucleotide encoding a repressor,
wherein the repressor, when present, represses the activity of the inducible pol III promoter; and
wherein a constitutive promoter is operably linked to the detectable marker polynucleotide and the repressor polynucleotide, wherein the detectable marker polynucleotide is linked downstream of the repressor polynucleotide via an internal ribosome entry site, thereby allowing for transcription of a polycistronic RNA encoding the repressor and the detectable marker.

32. (canceled)
